(19) United States
(12) Patent Application Publication
(10) Pub. No.: US 2012/0083645 A1
(43) Pub. Date: Apr. 5, 2012

(54) ECG-CORRELATED RADIOTHERAPY

(75) Inventors: Mingshan Sun, Sunnyvale, CA (US); Josh Star-Lack, Palo Alto, CA (US)

(73) Assignee: VARIAN MEDICAL SYSTEMS, INC., Palo Alto, CA (US)

(21) Appl. No.: 12/896,846

(22) Filed: Oct. 2, 2010

(51) Int. Cl. A61N 5/00; A61N 5/10

(52) U.S. Cl. 600/1; 378/65

(57) ABSTRACT

A method of delivering radiation includes obtaining a first signal that represents a cardiac state of the subject, and generating a control signal using a processor to operate a component of a treatment radiation machine based at least in part on the first signal. A method of delivering radiation includes determining a cardiac state of the subject, and generating a control signal using a processor to operate a component of a treatment radiation machine based at least in part on the determined cardiac state.

### FIELD

This invention relates to systems and methods of radiotherapy, and more specifically, to systems and methods for performing radiotherapy to treat tissue that is close to the heart, such as breast tissue, lung tissue, etc.

### BACKGROUND

Radiation therapy has been employed to treat tumorous tissue. In radiation therapy, a high energy beam is applied from an external source towards the patient. The external source, which may be rotating (as in the case for arc therapy), produces a collimated beam of radiation that is directed into the patient to the target site. The dose and placement of the dose must be accurately controlled to ensure that the tumor receives sufficient radiation, and that damage to the surrounding healthy tissue is minimized.

When using radiation to treat tissue (such as breast tissue, lung tissue, etc.) close to the heart, the heart may receive some radiation dose. Applicant determines that radiation dose received by a heart may increase the risk of mortality from ischemic heart disease. Thus, Applicant determines that it would be desirable to have a new system and method for performing radiotherapy in a way that would prevent or at least reduce cardiotoxicity at the heart from radiation dose.

Also, when using radiation to treat tissue close to the heart, the tissue may move according to the cardiac motion. Applicants determine that the targeting precision may increase if the radiation beam is synchronized to the heart beat.

### SUMMARY

In accordance with some embodiments, a method of delivering radiation includes obtaining a first signal that represents a cardiac state of the subject, and generating a control signal using a processor to operate a component of a treatment radiation machine based at least in part on the first signal.

In accordance with other embodiments, a system for delivering radiation includes a process configured for obtaining a first signal that represents a cardiac state of the subject, and generating a control signal to operate a component of a treatment radiation machine based at least in part on the first signal.

In accordance with other embodiments, a computer product includes a non-transitory medium storing a set of instructions, an execution of which causes a method to be performed, the method comprising obtaining a first signal that represents a cardiac state of the subject, and generating a control signal to operate a component of a treatment radiation machine based at least in part on the first signal.

In accordance with other embodiments, a method of delivering radiation includes determining a cardiac state of the subject, and generating a control signal using a processor to operate a component of a treatment radiation machine based at least in part on the determined cardiac state.

In accordance with other embodiments, a system for delivering radiation includes a processor configured for determining a cardiac state of the subject, and generating a control signal to operate a component of a treatment radiation machine based at least in part on the determined cardiac state.

In accordance with other embodiments, a computer product includes a non-transitory medium storing a set of instructions, an execution of which causes a method to be performed, the method comprising determining a cardiac state of the subject, and generating a control signal to operate a component of a treatment radiation machine based at least in part on the determined cardiac state.

Other and further aspects and features will be evident from reading the following detailed description of the embodiments, which are intended to illustrate, not limit, the invention.

### BRIEF DESCRIPTION OF THE DAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

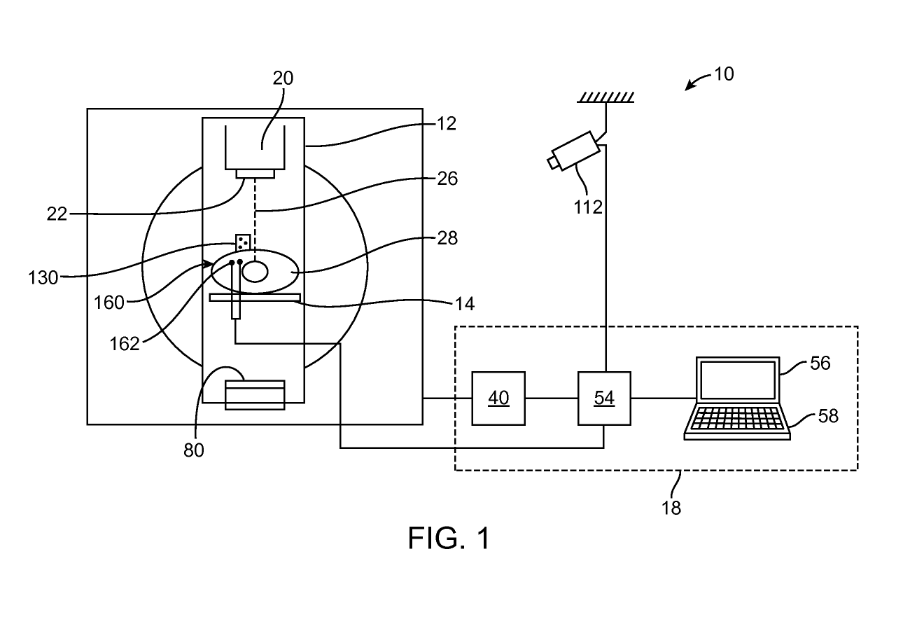

FIG. 1 illustrates a radiation system in accordance with some embodiments;

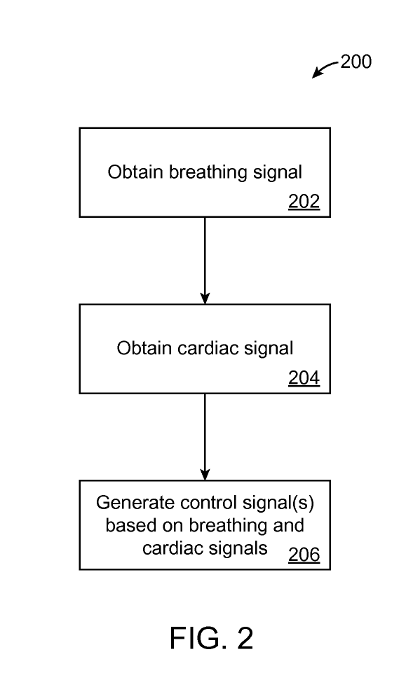

FIG. 2 illustrates a method of delivering radiation in accordance with some embodiments;

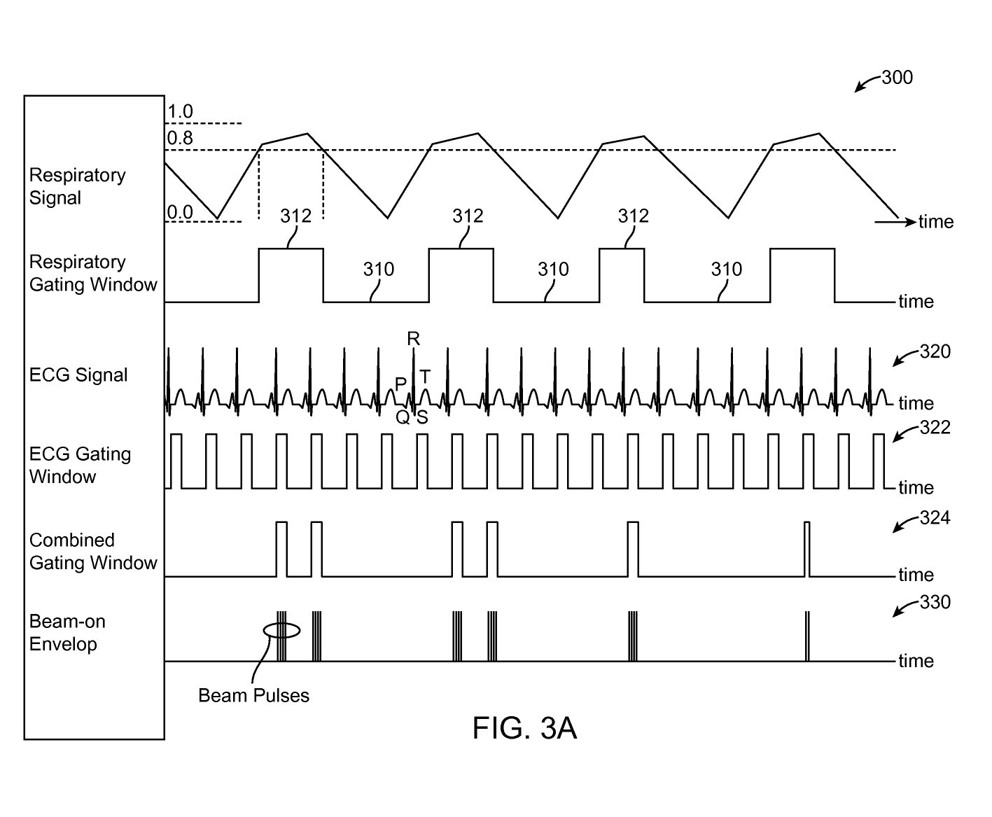

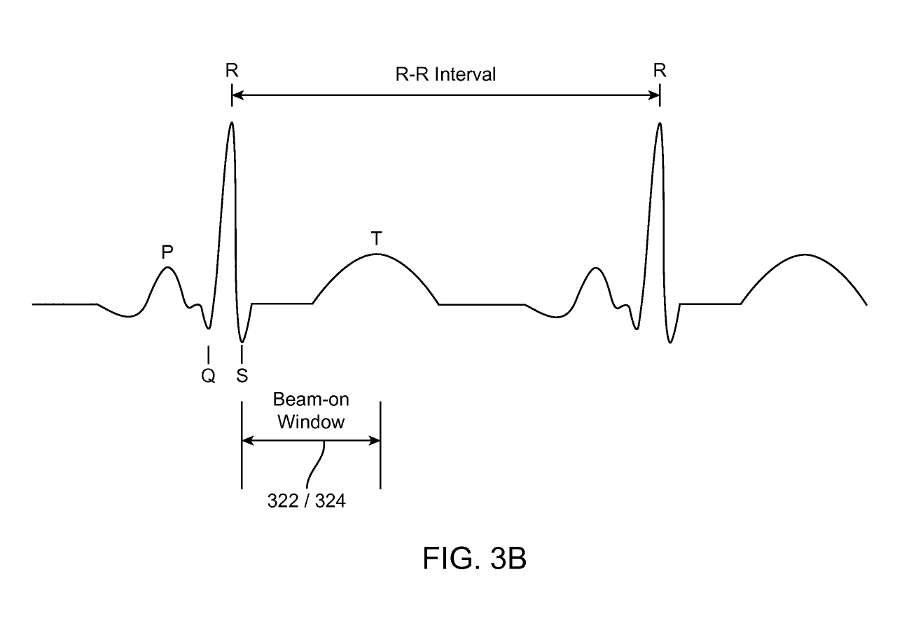

FIGS. 3A-3B illustrate a technique of delivering radiation based on breathing amplitude and cardiac state;

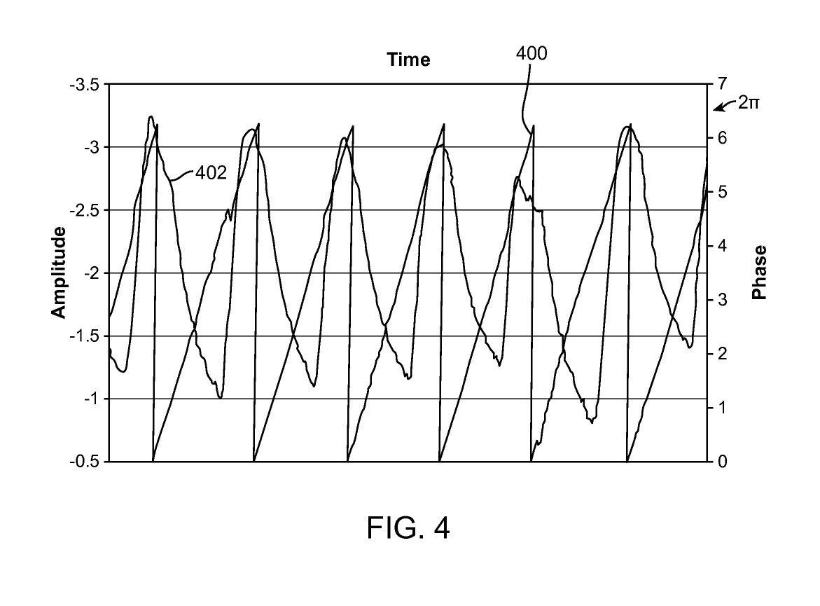

FIG. 4 is an exemplary chart showing phase and amplitude for a periodic signal;

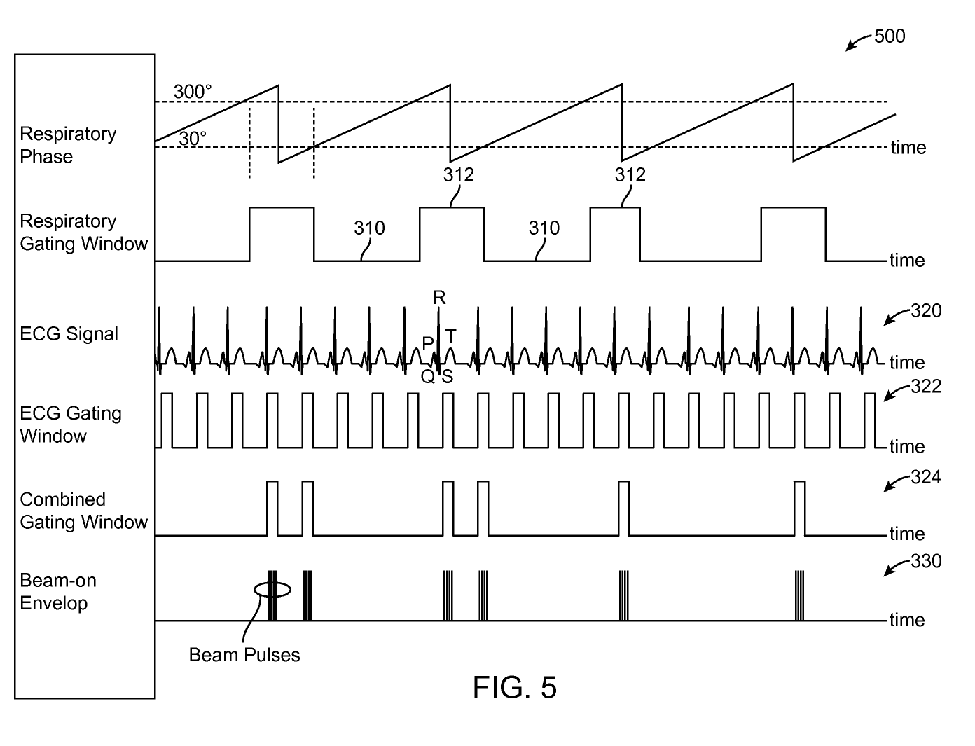

FIG. 5 illustrates a technique of delivering radiation based on breathing phase and cardiac state; and

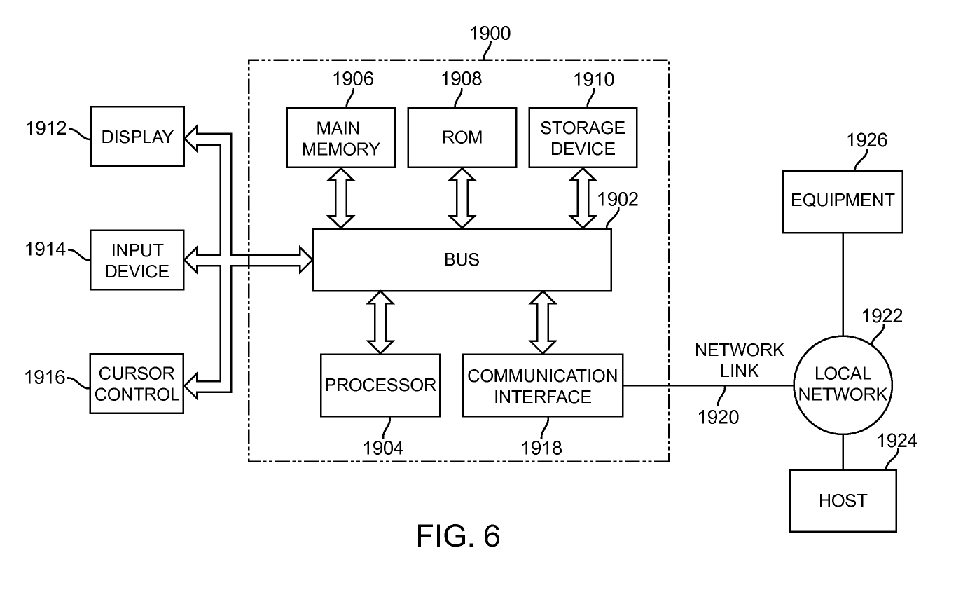

FIG. 6 is a block diagram of a computer system architecture, with which embodiments described herein may be implemented.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

FIG. 1 illustrates a radiation system 10 in accordance with some embodiments. The system 10 is a treatment system that includes a gantry 12, a patient support 14 for supporting a patient 28, and a control system 18 for controlling an operation of the gantry 12. The gantry 12 is in a form of a c-arm. The system 10 also includes a radiation source 20 that projects a beam 26 of radiation towards the patient 28 while the patient 28 is supported on support 14, and a collimator system 22 for controlling a delivery of the radiation beam 26. The radiation source 20 can be configured to generate a cone beam, a fan beam, or other types of radiation beams in different embodiments.

In the illustrated embodiments, the radiation source 20 is a treatment radiation source for providing treatment energy. In other embodiments, in addition to being a treatment radiation source, the radiation source 20 can also be a diagnostic radiation source for providing diagnostic energy for imaging purpose. In such cases, the system 10 will include an imager, such as the imager 80, located at an operative position relative to the source 20 (e.g., under the support 14). In some embodiments, the treatment energy is generally those energies of 160 kilo-electron-volts (keV) or greater, and more typically 1 mega-electron-volts (MeV) or greater, and diagnostic energy is generally those energies below the high energy range, and more typically below 160 keV. In other embodiments, the treatment energy and the diagnostic energy can have other energy levels, and refer to energies that are used for treatment and diagnostic purposes, respectively. In some embodiments, the radiation source 20 is able to generate X-ray radiation at a plurality of photon energy levels within a range anywhere between approximately 10 keV and approximately 20 MeV. In further embodiments, the radiation source 20 can be a diagnostic radiation source. In the illustrated embodiments, the radiation source 20 is coupled to the arm gantry 12. Alternatively, the radiation source 20 may be located within a bore.

It should be noted that the system 10 is not limited to providing treatment radiation in the form of x-ray, and that the system 10 may be configured to provide other form of treatment radiation. For example, in other embodiments, the radiation source 20 of the system 10 may be configured to provide radiation having photons, protons, carbon, or other charged particles.

In the illustrated embodiments, the control system 18 includes a processor 54, such as a computer processor, coupled to a control 40. The control system 18 may also include a monitor 56 for displaying data and an input device 58, such as a keyboard or a mouse, for inputting data. The operation of the radiation source 20 and the gantry 12 are controlled by the control 40, which provides power and timing signals to the radiation source 20, and controls a rotational speed and position of the gantry 12, based on signals received from the processor 54. Although the control 40 is shown as a separate component from the gantry 12 and the processor 54, in alternative embodiments, the control 40 can be a part of the gantry 12 or the processor 54.

As shown in FIG. 1, the system 10 also includes a breathing monitoring system 110 and a cardiac monitoring system 160. The breathing monitoring system 110 includes a camera 112 communicatively coupled to the processor 54. The camera 112 is oriented to view towards a patient 28. In the illustrated embodiments, a marker block 130 is placed on the patient 28, and the camera 112 is positioned so that it can view the marker block 130. The processor 54 is configured to process image signals (an example of breathing signals) from the camera 112, and process the image signals to thereby monitor the breathing of the patient 28. In some embodiments, the image signals and/or results of the processing of the image signals may be displayed on the monitor 56, for allowing a user to view them. Also, in some embodiments, the user may use the input device 58 to input parameters for processing of the image signals. In some embodiments, the processor 54, or at least part of the processor 54, may be considered to be a part of the breathing monitoring system 110. In other embodiments, the breathing monitoring system 110 may have its own processor that is different from the processor 54 for the system 10.

As shown in the figure, the marker block 130 includes a plurality of markers 132 that are viewable by the camera 112. Each marker 132 may include a reflective material so that it can be more easily detected by the camera 112. In the illustrated embodiments, the relative positions among the markers 132 are predetermined. The processor 54 is configured to determine the position of the marker block 130 using the predetermined relative positions of the markers 132. In particular, the processor 54 is configured to compare the pattern of the markers 132 in the image provided by the camera 112 with the known pattern of the markers 132 based on the predetermined relative positions of the markers 132. Based on the comparison, the processor 54 then determines the position of the marker block 130. By continuously processing image signals and determining the position of the marker block 130, the processor 54 can determine the breathing amplitude of the patient 28 in substantially real time.

In some embodiments, when using the system 10 of FIG. 1, the radiation source 20 is rotated about the patient 28 to deliver treatment radiation from a plurality of gantry angles, for example, as in arc therapy. As treatment radiation is being delivered to the patient 28, the breathing monitoring system 100 may be used to monitor the breathing of the patient 28. In some embodiments, the processor 54 processes the signals from the camera 112 to determine breathing amplitudes of the patient 28, and then gates the delivery of the treatment radiation based on the amplitudes. For example, the processor 54 may cause the radiation source 20 to deliver radiation, or to stop a delivery of radiation, when the determined amplitude is within a prescribed amplitude range. In other embodiments, the processor 54 processes the signals from the camera to determine respiratory phases of the patient 28, and then gates the delivery of the treatment radiation based on the respiratory phases. For example, the processor 54 may cause the radiation source 20 to deliver radiation, or to stop a delivery of radiation, when the determined phase is within a prescribed phase range. In other embodiments, instead of, or in addition to, controlling the delivery of radiation, the processor 54 may be configured to control the gantry 12 (e.g., stop, accelerate, or decelerate the gantry 12), and/or to adjust the collimator (e.g., leaves of the collimator), and/or to position the patient support 14, based on the determined amplitude and/or phase.

During the treatment process, the processor 54 monitors the patient's 28 breathing, and correlates feature(s) of the breathing (such as breathing signals, breathing amplitudes, breathing phases, etc.) with positions of the internal target region that is being irradiated by the radiation beam 26. For example, based on images received from the camera 112, the processor 54 then determines the phase/amplitude of the breathing cycle. The phase of the breathing cycle or the amplitude is then used by the processor 54 to determine a position of the internal target region based on a pre-established relationship between breathing phase/amplitude and position of the internal target region. In some embodiments, the relationship between the breathing phase/amplitude and target position may be pre-determined by a physician during a treatment planning process. For example, during a treatment planning process, it may be determined that when a patient is at breathing phase=40°, the corresponding position of the internal target region is at position X=45 mm, Y=23 mm, and Z=6 mm relative to the isocenter. This technique allows the treatment radiation system 10 to target delivery of radiation towards the target region based on breathing signals obtained by the system 10. Thus, it has the benefit of obviating the need to continuously or periodically imaging the internal target region using radiation, which may be harmful to the patient.

In the above embodiments, the system 110 is described as having a camera 112 for obtaining image signals that can be used to determine breathing amplitudes. In other embodiments, the system 110 may not include the camera 112. Instead, the system 110 may include other types of devices for providing breathing information. For example, in other embodiments, the system 110 may include a strain-gauge that is coupled to the patient 28. In such cases, the strain-gauge is communicatively coupled to the processor 54 for providing signals that represent breathing amplitudes of the patient 28. In other embodiments, the system 110 may include a sensor coupled to the patient's mouth and/or nose for sensing the breathing of the patient 28. The processor 54 is communicatively coupled to the sensor, and receives signals from the sensor. The signals may represent the breathing amplitudes, or may be used to obtain breathing amplitudes and/or breathing phases. In further embodiments, internal target tracking may be employed that uses radio frequency transponder(s) implanted in or near the target region (e.g., tumor). The transponder(s) is localized by an external array antenna transmitting query signals and processing the transponder response signals. In further embodiments, internal reference marker(s) such as bead(s) may be implanted, and imaged under x-ray fluoroscopic procedure. The x-ray image is then used to determine the breathing signals. The x-ray image may also be used to confirm and update the correlation between breathing state and known target tissue (e.g., breast, lung, etc.) position, and or the correlation between cardiac state and known target tissue position. In further embodiments, a markerless system may be used to determine the breathing state. For example, the camera or another imaging system may be used to obtain images of at least a part of the patient, and the image may then be processed by the processor to determine the breathing state. Other types of breathing sensing devices may be used with the processor 54 in other embodiments.

As shown in FIG. 1, the cardiac monitoring system 160 includes one or more electrodes 162 that are coupled to the patient 28. The electrode(s) is communicatively coupled to the processor 54. In some embodiments, the processor 54, or at least part of the processor 54, may be considered to be a part of the cardiac monitoring system 160. In other embodiments, the cardiac monitoring system 160 may have its own processor that is different from the processor 54 for the system 10. Also, in further embodiments, the cardiac monitoring system 160 and the breathing monitoring system 110 may have a common processor that is different from the processor 54 for the radiation system 10. In some embodiments, the cardiac monitoring system 160 may be implemented using a ECG device.

FIG. 2 illustrates a method 200 of delivering radiation in accordance with some embodiments. In the illustrated embodiments, the method 200 is performed by the system 10 (e.g., processor 54) of FIG. 1. In other embodiments, the method 200 may be performed by another processor, which may be a part of the breathing monitoring system 110 and the cardiac monitoring system 160. In further embodiments, the method 200 may be performed by a combination of processors that are respective components of the monitoring systems 110, 160.

First, breathing signal of the patient is obtained (step 202). In some embodiments, such may be accomplished by the processor 54 receiving image signals from the camera 112, wherein the image signals themselves may be considered breathing signal(s). In other embodiments, image signals are processed by the processor 54 to determine breathing amplitude. In such cases, the breathing amplitude may be considered the breathing signal, and the act of obtaining the breathing signal may be accomplished by processing the image signals to determine breathing amplitude using the processor 54. In other embodiments, the breathing signal may be obtained using other devices that are capable of measuring a parameter representative of a breathing state. In further embodiments, the act of obtaining the breathing signal may be performed by the processor 54 receiving an input from the user, who visually determines the breathing state (e.g., end of inhalation, end of exhalation, etc.) of the patient. Also, in any of the embodiments described herein, the user may instruct the patient 28 to control his/her breathing (e.g., to breath hold). In such cases, when the patient 28 has achieved the requested breathing state, the user then sends an input (e.g., using the input device 58) to the processor 54, indicating that a prescribed breathing state has been achieved.

Next, the cardiac signal of the patient is obtained (step 204). In the illustrated embodiments, such may be accomplished by the processor 54 receiving ECG signals via the electrodes 162.

Next, the processor 54 is configured to generate control signal(s) to operate component(s) of the radiation system 10 based on the breathing signal and/or the cardiac signal (step 206). In some embodiments, when the processor 54 determines that the breathing signal (state) is at a prescribed breathing value or within a prescribed breathing state range, and the cardiac signal (state) is at a prescribed cardiac value or within a prescribed cardiac state range, then the processor 54 generates one or more control signals to operate the radiation system 10. For example, the processor 54 may generate control signal(s) to activate or deactivate the radiation source 20, move the leaves of the collimator 22, control motion of the gantry 12 (e.g., accelerate, decelerate, stop, etc.), and/or position the patient support 14. In some embodiments, the prescribed breathing value or breathing state range, and the prescribed cardiac value or cardiac state range, are selected such that they correspond with the condition in which the heart's distance from the chest wall is the furthest. This condition may be determined to be achieved when the breathing state is at or near the end of an inhalation, and when the cardiac state is at the end of the systolic phase (e.g., the ST segment when the ventricles contract). The ventricular systole is the contraction of the myocardium of the left and right ventricles. In some embodiments, the prescribed cardiac state range (cardiac gating window) may start at the S point and stop at the apex of the T wave (see FIG. 3). In other embodiments, the cardiac state range may begin and/or stop at other positions along the cardiac signal. In the illustrated embodiments, the processor 54 may be configured to generate a control signal to activate the radiation source 20 to deliver treatment radiation. In one implementation, the control signal causes the radiation source 20 to deliver beam pulses at a rate of 360 pulses per second. If the cardiac gating window is 200 ms in length, then there will be about 70 pulses in the cardiac gating window. In some embodiments, the prescribed breathing value or prescribed breathing state range, and the prescribed cardiac value or prescribed cardiac state range may be input by a user using the input device 58.

Although the method 200 is illustrated as obtaining the breathing signal before the cardiac signal, in other embodiments, the cardiac signal may be obtained before the breathing signal. In further embodiments, the breathing signal and the cardiac signal may be obtained independent of each other (e.g., they may be obtained simultaneously).

As discussed, the delivery of treatment radiation may be based on a determined breathing state and cardiac state. In some embodiments, the breathing state may be represented by a breathing amplitude. FIGS. 3A-3B illustrate a technique of delivering treatment radiation based on breathing amplitude (an example of a breathing state) and cardiac state. As shown in the example, when the processor 54 determines that the breathing amplitude is at a prescribed amplitude or within a prescribed breathing amplitude range (between 0.8 and 1.0 in the illustrated example), and the cardiac cycle is at a prescribed cardiac state, then the processor 54 generates one or more control signals to activate the radiation source 20. In particular, when the breathing amplitude 300 is between 0.8 and 1.0 in the example, the processor 54 then permits radiation to be delivered within the beam on envelops 312 (i.e., within respiratory gating window). Otherwise, radiation is not allowed to be delivered during beam hold 310 (i.e., outside the respiratory gating window). In other embodiments, the range of amplitudes for gating may be different from the example discussed. At the same time, the processor 54 monitors the ECG signals 320. In particular, the processor 54 determines from the ECG signals 320 whether the cardiac state is at the prescribed cardiac state (e.g., whether the ECG signal is within a cardiac state range), as represented by the ECG gating window 322. When the processor 54 determines that the breathing amplitude is within the respiratory gating window 312, and that the ECG signal is within the ECG gating window 322, then the processor 54 generates one or more control signals to cause the radiation beam to be delivered. The periods in which the radiation beams are delivered are represented by the combined gating window 324, which is obtained by combining the respiratory gating window 312 with the ECG gating window 322. Because radiation will be delivered at prescribed breathing state and at prescribed cardiac state, the delivery of radiation will result in a series of beam pulses 330 (only within the respiratory gating windows 312) that are synchronized with the cardiac cycle (ECG signals).

In the illustrated embodiments, the prescribed amplitude or amplitude range may be selected to be that associated with the breathing state that is at or near the end of inhalation. In some embodiments, the breathing state is considered to be near the end of an inhalation when the breathing amplitude is anywhere between 50% and 100% of the maximum amplitude, or more preferably, anywhere between 80% and 100% of the maximum amplitude (as illustrated in the above example), with 100% representing the end of inhalation (maximum breathing amplitude). The prescribed breathing amplitude range may have other ranges in other embodiments.

In other embodiments, the delivery of treatment radiation may be based on breathing phase (another example of a breathing state) and cardiac state. The phase of a physiological cycle represents a degree of completeness of a physiological cycle. In some embodiments, the phases of a respiratory cycle may be represented by a phase variable having values between 0° and 360°. FIG. 4 illustrates an example of a phase diagram 400 that is aligned with a corresponding amplitude/position diagram 402. Amplitude diagram 402 includes positional points of the marker block 130 determined using embodiments of the technique described herein. In the illustrated example, a phase value of 0° (and 360°) represents a peak of an inhale state, and the phase value varies linearly between 0° and 360° in a physiological cycle. Thus, for each breathing amplitude, the processor 54 can determine the corresponding phase of the respiratory cycle. In some embodiments, the determined phase may be considered an example of a breathing signal. In such cases, the act of determining the phase by the processor 54 may be performed in step 202 to obtain the breathing signal.

FIG. 5 illustrates a technique of delivering treatment radiation based on breathing phase and cardiac state. As shown in the example, when the processor 54 determines that the breathing phase is at a prescribed phase (e.g., at or near the end of inhalation) or within a prescribed breathing phase range, and the cardiac cycle is at a prescribed cardiac state, then the processor 54 generates one or more control signals to activate the radiation source 20. In the illustrated example, the peak of inhalation is represented by phase value of 360° (or 0°). In the example, when the breathing phase 500 is between 300° and 360° (representing rising of the chest to reach the peak of inhalation) and between 0° and 30° (representing lowering of the chest right after the peak of inhalation), the processor 54 then permits radiation to be delivered within the beam on envelops (respiratory gating window) 312. Otherwise, radiation is not allowed to be delivered during beam hold 310 (i.e., outside the respiratory gating window). At the same time, the processor 54 monitors the ECG signals 320. In particular, the processor 54 determines from the ECG signals 320 whether the cardiac state is at the prescribed cardiac state (e.g., whether the ECG signal is within a cardiac state range), as represented by the ECG gating window 322. When the processor 54 determines that the breathing phase is within the respiratory gating window 312, and that the ECG signal is within the ECG gating window 322, then the processor 54 generates one or more control signals to cause the radiation beam to be delivered. The periods in which the radiation beams are delivered are represented by the combined gating window 324, which is obtained by combining the respiratory gating window 312 with the ECG gating window 322. Because radiation will be delivered at prescribed breathing state and at prescribed cardiac state, the delivery of radiation will result in a series of beam pulses 330 (only within the respiratory gating windows 312) that are synchronized with the cardiac cycle (ECG signals).

In the above example, the breathing state is considered to be near the end of the inhalation when the breathing phase is anywhere between 300° and 360° and anywhere between 0° and 30°. In other embodiments, the breathing state is considered to be near the end of an inhalation when the breathing phase is anywhere between 270° and 360° (2π), or more preferably, anywhere between 300° and 360°, with 360° representing the end of inhalation. The prescribed breathing phase may have other phase ranges in other embodiments.

As illustrated in the above embodiments, using cardiac motion to gate the delivery of radiation is advantageous because it allows radiation to be delivered when the distance between the heart and the chest wall is the maximum. In the case in which the radiation is used to treat breast tissue, the above technique allows radiation to be delivered to the breast tissue while preventing (or at least reducing or minimizing) radiation that is delivered to the heart. This is because when the patient is in deep inhalation, the chest wall is relatively further away from the heart, and when the patient's heart is in the systolic phase, at least a portion of the heart is further away from the chest. It should be noted that in some embodiments, the distance between the heart and the chest wall is considered the “maximum” when the distance is above a prescribed distance. Thus, the distance between the heart and the chest wall does not need to be at the highest degree in order for the distance to be considered a “maximum” distance. In other embodiments, the distance is considered a “maximum” distance when the distance is at the highest degree.

Also, in the case in which the radiation is used to treat lung tissue, or other tissue that is close to the heart (such as the chest wall), the above technique allows radiation to be precisely delivered to the target tissue. The above technique may also allow radiation to be delivered to the target tissue while preventing (or at least reducing or minimizing) radiation that is delivered to the heart.

In other embodiments, the prescribed cardiac state for delivering radiation may be selected when the heart motion is small (i.e. during diastole which corresponds to the T-wave when heart motion is smallest). In some cases, the heart motion is considered “small” when the position of a target tissue does not change by more than 20%, and more preferably does not change by more than 10%, due to the heart motion. Similarly, the breathing state for allowing radiation to be delivered may be selected when the breathing motion is small (e.g., near end of inhale phase, or near end of exhale phase, when the breathing motion is the smallest). In some cases, the breathing motion is considered “small” when the position of a target tissue does not change by more than 20%, and more preferably does not change by more than 10%, due to the breathing motion. Such technique allows radiation to be delivered to target tissue at a time when the position of the target tissue is the least affected by motion due to breathing and heart beating. Since the T wave may be difficult to be used as the basis for gating the delivery of radiation, in some embodiments, the diastole phase can be inferred using a prospective technique that assumes a given time between the most recent R-wave and the next R-wave. In such technique, cardiac gating may occur at a time spanning 50-70% of the R-R interval.

As discussed, in some embodiments, the patient 28 may be instructed to control his/her breathing during a treatment session. In one implementation, the patient 28 is instructed to hold his /her breath at the end of deep-inhalation. The patient 28 is monitored with ECG, which feeds the ECG signal to the processor 54. The processor 54 then synchronizes the delivery of beam pulses with the ECG signal, along with the breath hold. When the ECG signal indicates that the cardiac state is at the systolic phase (e.g., end of systolic phase) or another prescribed phase, then the processor 54 generates a control signal to activate the beam. Thus, the delivery of beam pulse is synchronized with the ECG signal. The beam is on during a short period when the cardiac cycle is at the end of the systolic phase. Such technique results in radiation being delivered to the patient 28 when the distance between the heart and the chest wall is the largest. In particular, the distance is largest because the breath hold at deep inhalation results in the chest wall being furthest away from the heart, and because at the systolic phase, the heart contracts furthest away from the chest wall.

It should be noted that because delivering radiation based on breathing state and cardiac state results in pulses of radiation beams that are delivered at the prescribed cardiac state, the treatment duty cycle may be reduced and the treatment time may increase. In some embodiments, the treatment speed may be improved by increasing flux using, for example, flattening filter-free treatment schemes. A radiation beam exhibits a Gaussian-shaped intensity distribution across its field. A flattening filter, which has a conical shape, may be used to compensate for this effect to make the beam more uniform. This is sometimes needed for radiotherapy where only a very few fields were used, and for conformal radiotherapy. With IMRT and arc therapy, this effect can be accounted for in planning. In particular, by removing this filter, the beam intensity is much increased, so the beam-on time can be reduced.

In other embodiments, the gun timing (e.g., the timing of actuating the radiation source) may be adjusted to create bursts of pulses during the treatment. Another technique for increasing the treatment duty cycle is to track the tumor as function of respiratory phase (i.e., the aiming of the beam follows the movement of tumor due to respiration), but the gating of the delivery of radiation is performed as a function of cardiac phase. In further embodiments, the breathing state and the cardiac state may be used to aim the radiation source so that it follows the target to compensate for both the breathing and cardiac motions. In such cases, the beam pulses may be more frequent, and may even be substantially continuous.

In any of the embodiments described herein, the breathing signals obtained in step 202, and/or the cardiac signals obtained in step 204, may be used to predict signals for a future time. In such cases, in step 202, the processor 54 receives a current breathing signal, and predicts a value for a future breathing signal at a future time. For example, the processor 54 may be configured to determine a pattern of breathing based on past respiratory cycles. In such cases, the processor 54 then uses the determined breathing pattern to predict what the breathing state will be at a future time (e.g., a prescribed Δt from the current time) based on the current breathing state.

Similarly, in step 204, the processor 54 receives a current cardiac signal, and predicts a value for a future cardiac signal at the future time. For example, the processor 54 may be configured to determine a pattern of cardiac motion based on past cardiac cycles. In such cases, the processor 54 then uses the determined cardiac motion pattern to predict what the cardiac state will be at a future time (e.g., a prescribed Δt from the current time) based on the current cardiac state.

In step 206, the processor 54 is configured to operate component(s) of the radiation system 10 based on the predicted breathing signal and/or the predicted cardiac signal. In some embodiments, if the processor 54 determines that the predicted breathing signal (state) is at a prescribed breathing value or within a prescribed breathing state range, and the predicted cardiac signal (state) is at a prescribed cardiac value or within a prescribed cardiac state range, then the processor 54 generates one or more control signals to operate the radiation system 10. For example, the processor 54 may generate control signal(s) to activate or deactivate the radiation source 20, move the leaves of the collimator 22, control motion of the gantry 12 (e.g., accelerate, decelerate, stop, etc.), and/or position the patient support 14. In some embodiments, the prescribed breathing value or prescribed breathing state range, and the prescribed cardiac value or prescribed cardiac state range, are selected such that they correspond with the condition in which the heart's distance from the chest wall is the furthest. This condition may be determined to be achieved when the breathing state is at or near the end of an inhalation, and when the cardiac state is at the end of the systolic phase (e.g., the ST segment when the ventricles contract). In such cases, the processor 54 may be configured to generate a control signal to activate the radiation source 20 to deliver treatment radiation. In some embodiments, the prescribed breathing value or breathing state range, and the prescribed cardiac value or cardiac state range may be input by a user using the input 58.

Using predicted breathing state and/or the predicted cardiac state to operate the radiation system 10 is advantageous because it allows latency of the various components to be compensated. For example, if there is significant latency resulted from the processing of the breathing signal, processing of the cardiac signal, activation or deactivation of the radiation source 20, movement of the leaves of the collimator 22, movement of the gantry 12, and/or movement of the patient support 14, the predicted state(s) may be used to predictively control the system 10 so that the latency of any of these components may be compensated. In further embodiments, the predicted state(s) may be used to predictively control the system 10 to track a target, such as the tumor.

Also, although the above embodiments have been described with reference to considering both the breathing state and the cardiac state in a radiation treatment procedure, in other embodiments, the radiation treatment procedure may consider only the cardiac state and not the breathing state. For examples, in some treatment procedures to treat breast or lung tissue, the gating of the radiation beam may be based on the cardiac state only. The breathing motion may be compensated by instructing the patient to perform breath-hold, or by configuring the system to aim the radiation source so that it follows the breathing motion. In such cases, the embodiments of the system 10 described herein do not include or use any device for determining a breathing state of the patient, and embodiments of the method 200 described herein do not include the act of determining a breathing signal.

Computer System Architecture

FIG. 6 is a block diagram that illustrates an embodiment of a computer system 1900 upon which an embodiment of the invention may be implemented. Computer system 1900 includes a bus 1902 or other communication mechanism for communicating information, and a processor 1904 coupled with the bus 1902 for processing information. The processor 1904 may be an example of the processor 54 of FIG. 1, or another processor that is used to perform various functions described herein. In some cases, the computer system 1900 may be used to implement the processor 14 (or other processors described herein). The computer system 1900 also includes a main memory 1906, such as a random access memory (RAM) or other dynamic storage device, coupled to the bus 1902 for storing information and instructions to be executed by the processor 1904. The main memory 1906 also may be used for storing temporary variables or other intermediate information during execution of instructions to be executed by the processor 1904. The computer system 1900 further includes a read only memory (ROM) 1908 or other static storage device coupled to the bus 1902 for storing static information and instructions for the processor 1904. A data storage device 1910, such as a magnetic disk or optical disk, is provided and coupled to the bus 1902 for storing information and instructions.

The computer system 1900 may be coupled via the bus 1902 to a display 1912, such as a cathode ray tube (CRT) or a flat panel, for displaying information to a user. An input device 1914, including alphanumeric and other keys, is coupled to the bus 1902 for communicating information and command selections to processor 1904. Another type of user input device is cursor control 1916, such as a mouse, a trackball, or cursor direction keys for communicating direction information and command selections to processor 1904 and for controlling cursor movement on display 1912. This input device typically has two degrees of freedom in two axes, a first axis (e.g., x) and a second axis (e.g., y), that allows the device to specify positions in a plane.

The computer system 1900 may be used for performing various functions (e.g., calculation) in accordance with the embodiments described herein. According to one embodiment, such use is provided by computer system 1900 in response to processor 1904 executing one or more sequences of one or more instructions contained in the main memory 1906. Such instructions may be read into the main memory 1906 from another computer-readable medium, such as storage device 1910. Execution of the sequences of instructions contained in the main memory 1906 causes the processor 1904 to perform the process steps described herein. One or more processors in a multi-processing arrangement may also be employed to execute the sequences of instructions contained in the main memory 1906. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to the processor 1904 for execution. Such a medium may take many forms, including but not limited to, non-volatile media, volatile media, and transmission media. Non-volatile media includes, for example, optical or magnetic disks, such as the storage device 1910. A non-volatile medium may be considered as an example of a non-transitory medium. Volatile media includes dynamic memory, such as the main memory 1906. A volatile medium may be considered as another exampler of a non-transitory medium. Transmission media includes coaxial cables, copper wire and fiber optics, including the wires that comprise the bus 1902. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to the processor 1904 for execution. For example, the instructions may initially be carried on a magnetic disk of a remote computer. The remote computer can load the instructions into its dynamic memory and send the instructions over a telephone line using a modem. A modem local to the computer system 1900 can receive the data on the telephone line and use an infrared transmitter to convert the data to an infrared signal. An infrared detector coupled to the bus 1902 can receive the data carried in the infrared signal and place the data on the bus 1902. The bus 1902 carries the data to the main memory 1906, from which the processor 1904 retrieves and executes the instructions. The instructions received by the main memory 1906 may optionally be stored on the storage device 1910 either before or after execution by the processor 1904.

The computer system 1900 also includes a communication interface 1918 coupled to the bus 1902. The communication interface 1918 provides a two-way data communication coupling to a network link 1920 that is connected to a local network 1922. For example, the communication interface 1918 may be an integrated services digital network (ISDN) card or a modem to provide a data communication connection to a corresponding type of telephone line. As another example, the communication interface 1918 may be a local area network (LAN) card to provide a data communication connection to a compatible LAN. Wireless links may also be implemented. In any such implementation, the communication interface 1918 sends and receives electrical, electromagnetic or optical signals that carry data streams representing various types of information.

The network link 1920 typically provides data communication through one or more networks to other devices. For example, the network link 1920 may provide a connection through local network 1922 to a host computer 1924 or to equipment 1926 such as a radiation beam source or a switch operatively coupled to a radiation beam source. The data streams transported over the network link 1920 can comprise electrical, electromagnetic or optical signals. The signals through the various networks and the signals on the network link 1920 and through the communication interface 1918, which carry data to and from the computer system 1900, are exemplary forms of carrier waves transporting the information. The computer system 1900 can send messages and receive data, including program code, through the network(s), the network link 1920, and the communication interface 1918.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. For example, the term “image” needs not be limited to an image that is displayed visually, and may refer to image data that is stored. Also, the term “processor” may include one or more processing units, and may refer to any device that is capable of performing mathematical computation implemented using hardware and/or software. The term “processor” may also refer to software stored in a non-transitory medium in other embodiments. The specification and drawings are, accordingly, to be regarded in an illustrative rather than restrictive sense. The present inventions are intended to cover alternatives, modifications, and equivalents, which may be included within the spirit and scope of the present inventions as defined by the claims.

What is claimed:

1. A method of delivering radiation, comprising:
obtaining a first signal that represents a cardiac state of the subject; and
generating a control signal using a processor to operate a component of a treatment radiation machine based at least in part on the first signal.

2. The method of claim 1, further comprising obtaining a second signal that represents a breathing state of a subject, wherein the control signal is generated to operate the component of the treatment radiation machine based at least in part on the first and second signals.

3. The method of claim 2, wherein the second signal is obtained by receiving an image from a camera.

4. The method of claim 2, wherein the second signal is obtained by processing one or more images, and determining a breathing phase.

5. The method of claim 2, wherein the second signal is obtained by processing one or more images, and determining a breathing amplitude.

6. The method of claim 2, wherein the act of generating the control signal is performed when the first signal indicates that the subject is in a systolic phase, and when the second signal indicates that the subject is at or near an end of an inhalation.

7. The method of claim 6, wherein the act of generating the control signal comprises generating the control signal to deliver radiation when the subject is at or near the end of inhalation, and when the subject is in the systolic phase.

8. The method of claim 2, wherein the act of generating the control signal comprises generating the control signal to deliver radiation when a breathing motion is small and when a cardiac motion is small.

9. The method of claim 2, wherein the second signal is used to control a radiation source of the treatment radiation machine so that an aiming of the radiation source follows a moving object due to breathing.

10. The method of claim 1, wherein the first signal is obtained by receiving a ECG signal from a ECG device.

11. The method of claim 1, wherein the control signal is generated to operate the component of the treatment radiation machine to treat cancerous breast tissue or cancerous lung tissue.

12. The method of claim 1, wherein the treatment radiation machine is configured to provide x-ray, photons, protons, carbon, or other charged particles.

13. A system for delivering radiation, comprising:
a process configured for:
obtaining a first signal that represents a cardiac state of the subject;
and
generating a control signal to operate a component of a treatment radiation machine based at least in part on the first signal.

14. The system of claim 13, wherein the processor is further configured for obtaining a second signal that represents a breathing state of a subject, wherein the processor is configured to generate the control signal to operate the component of the treatment radiation machine based at least in part on the first and second signals.

15. The system of claim 14, wherein the second signal comprises an image signal, a breathing phase, or a breathing amplitude.

16. The system of claim 14, wherein the processor is configured for generating the control signal when the first signal indicates that the subject is in a systolic phase, and when the second signal indicates that the subject is at or near an end of inhalation.

17. The system of claim 16, wherein the processor is configured for generating the control signal to deliver radiation when the subject is at or near the end of inhalation, and when the subject is in the systolic phase.

18. The system of claim 14, wherein the processor is configured for generating the control signal to deliver radiation when a breathing motion is small and when a cardiac motion is small.

19. The system of claim 14, wherein the processor is configured to control a radiation source of the treatment radiation machine based at least in part on the second signal, so that an aiming of the radiation source follows a moving object due to breathing.

20. The system of claim 13, wherein the processor is configured to obtain the first signal by receiving a ECG signal from a ECG device.

21. The system of claim 13, wherein the treatment radiation machine is configured to provide x-ray, photons, protons, carbon, or other charged particles.

22. A computer product having a non-transitory medium storing a set of instructions, an execution of which causes a method to be performed, the method comprising:
obtaining a first signal that represents a cardiac state of the subject; and
generating a control signal to operate a component of a treatment radiation machine based at least in part on the first signal.

23. A method of delivering radiation, comprising:
determining a cardiac state of the subject; and
generating a control signal using a processor to operate a component of a treatment radiation machine based at least in part on the determined cardiac state.

24. The method of claim 23, further comprising determining a breathing state of a subject, wherein the control signal is generated to operate the component of the treatment radiation machine based at least in part on the determined cardiac state and the determined breathing state of the subject.

25. The method of claim 24, wherein the act of generating the control signal is performed when the subject is at or near an end of exhalation or inhalation, and when the subject is in a systolic phase.

26. The method of claim 25, wherein the act of generating the control signal comprises generating the control signal to deliver radiation when the subject is at or near the end of exhalation or inhalation, and when the subject is in the systolic phase.

27. The method of claim 24, wherein the act of generating the control signal is performed to deliver radiation when a breathing motion is small and when a cardiac motion is small.

28. The method of claim 24, wherein the breathing state is used to control a radiation source of the treatment radiation machine so that an aiming of the radiation source follows a moving object due to breathing.

29. The method of claim 23, wherein the cardiac state is determined using a ECG signal from a ECG device.

30. The method of claim 23, wherein the control signal is generated to operate the component of the treatment radiation machine to treat breast tissue or lung tissue.

31. The method of claim 23, wherein the treatment radiation machine is configured to provide x-ray, photons, protons, carbon, or other charged particles.

32. A system for delivering radiation, comprising:
a processor configured for:
determining a cardiac state of the subject; and
generating a control signal to operate a component of a treatment radiation machine based at least in part on the determined cardiac state.

33. The system of claim 32, wherein the processor is further configured for determining a breathing state of a subject, wherein the control signal is generated to operate the component of the treatment radiation machine based at least in part on the determined cardiac state and the determined breathing state of the subject.

34. The system of claim 33, wherein the processor is configured to generate the control signal when the subject is at or near an end of exhalation or inhalation, and when the subject is in a systolic phase.

35. The system of claim 34, wherein the processor is configured to generate the control signal to deliver radiation when the subject is at or near the end of exhalation or inhalation, and when the subject is in the systolic phase.

36. The system of claim 33, wherein the processor is configured for generating the control signal to deliver radiation when a breathing motion is small and when a cardiac motion is small.

37. The system of claim 33, wherein the processor is configured to control a radiation source of the treatment radiation machine based at least in part on the breathing state, so that an aiming of the radiation source follows a moving object due to breathing.

38. The system of claim 32, wherein the processor is configured to determine the cardiac state using a ECG signal from a ECG device.

39. The system of claim 32, wherein the treatment radiation machine is configured to provide x-ray, photons, protons, carbon, or other charged particles.

40. A computer product having a non-transitory medium storing a set of instructions, an execution of which causes a method to be performed, the method comprising:
determining a cardiac state of the subject; and
generating a control signal to operate a component of a treatment radiation machine based at least in part on the determined cardiac state.
